(19) United States
(12) Patent Application Publication
(10) Pub. No.: US 2022/0347188 A1
(43) Pub. Date: Nov. 3, 2022

(54) PROMOTING TISSUE REGENERATION

(71) Applicant: FRIEDRICH MIESCHER INSITUTE FOR BIOMEDICAL RESEARCH, Basel (CH)

(72) Inventors: Prisca LIBERALI, Zurich (CH); Ilya LUKONIN, Lorrach (DE)

(21) Appl. No.: 17/622,608

(22) PCT Filed: Jun. 24, 2020
(86) PCT No.: PCT/IB2020/055959

§ 371 Date: Dec. 23, 2021

(30) Foreign Application Priority Data
Jun. 27, 2019 (EP) 19182782.3

(51) Int. Cl. A61K 31/5513; A61K 31/505; A61K 31/192; A61K 45/06; A61P 1/16; A61P 43/00

(52) U.S. Cl. CPC A61K 31/5513; A61K 31/505; A61K 31/192; A61K 45/06; A61P 1/16; A61P 43/00

(57) ABSTRACT

The present invention relates to the novel use of inhibitors of the retinoic acid pathway to increase tissue regeneration.

### TECHNICAL FIELD

The present disclosure relates to a novel use of antagonists of the retinoic acid pathway to promote tissue regeneration.

### BACKGROUND OF THE INVENTION

Intestinal organoids are an intestinal epithelium ex vivo culture system that recapitulates numerous important aspects of the parent tissue: cell type diversity, spatial organization, but also the ability to regenerate and return to homeostatic conditions following damage (Grun et al., 2015; Sato et al., 2011; Serra et al., 2019). In fact, an entire intestinal organoid can develop from a single cell forming an emergent, self-organized structure undergoing spatially and temporally controlled cell fate transitions (Sato et al., 2009; Serra et al., 2019). In the first step, single cells undergo several rounds of division forming a symmetric cyst-like structure. At this stage, all cells composing the cyst reside in a Yap-dependent regenerative state and lack expression of genes characteristic for intestinal cell types (Serra et al., 2019). Subsequently this symmetry is broken, ultimately resulting in the formation of secretory Paneth cells. Whereas in vivo the Wnt secreting function of Paneth cells can be taken over by the mesenchyme (Farin et al., 2012), Paneth cells are an indispensable Wnt source for maintaining the stem cell niche in the crypts in vitro (Sato et al., 2011). Once the crypt region is established, cells distal from the crypt begin to differentiate to enterocytes that make up the villi of the intestinal epithelium (Sato et al., 2009). Thus, development of intestinal organoids from a single cell mimics the regeneration of a whole tissue and the re-establishment of homeostasis and it relies on the crosstalk and coordination of numerous signaling pathways (Chacon-Martinez et al., 2018; Qi et al., 2017). Therefore, organoids offer a great platform to study functional interactions in a high content screening setting, enabling the comparison of thousands of arrayed perturbations in systematic, controlled assay conditions and mapping functional genetic interactions during organoid temporal and spatial self-organization.

The present inventors developed a novel image-based phenotypic screening platform in intestinal organoids cultured from single cells to generate a toolbox of compounds shaping the intestinal organoid phenotypic landscape. The compounds were used to infer functional genetic interactions and identify pathways impacting different steps of organoid development. The inventors focused on compounds that improve the regeneration potential of the intestinal epithelium and discovered a novel role for nuclear retinoic acid receptors during organoid damage response and homeostasis both in vitro and in vivo.

### SUMMARY OF THE INVENTION

Thus, using this high-content image-based screening assay to unravel molecular mechanisms orchestrating organoid formation and self-organization, the present inventors serendipitously found a crucial role of retinoic acid nuclear receptors in controlling the exit from the regenerative state and in driving differentiation.

The present invention thus provides an inhibitor of the retinoic acid pathway for use in a method of increasing tissue regeneration. This tissue can be epithelium, for instance intestinal epithelium. In other embodiments, the tissue can be liver.

The inhibitor of the retinoic acid pathway can be an antagonist of the RXR. For instance, the antagonist of the retinoic acid pathway can selected from the group comprising HX 531, PA452 and UVI3003. In an embodiment, the inhibitor of the retinoic acid pathway is a compound of formula (I) or a pharmaceutically acceptable salt thereof,

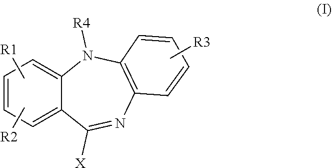

wherein R₁ and R₂, independently of each other, represent hydrogen, or C₁-C₇-alkyl, or R₁ and R₂ together with the carbon atoms of the phenyl ring to which they bind form a 5-, 6- or 7-membered cycloalkyl ring, which ring may optionally be substituted by one or more C₁-C₇-alkyl groups, which alkyl groups may also together form one or more 3-, 4-, 5-, 6- or 7-membered rings; R₃ represents —CN, —CO—R₅, or hydrogen, provided that, if R₃ is hydrogen, R₄ must represent C₃-C₇-alkenyl or C₃-C₇-alkynyl; R₅ represents aryl, or alkyl being unsubstituted or substituted by halogen, cyano, nitro, hydroxy, C₁-C₇-alkoxy, carboxyl or aryl; R₄ represents C₁-C₇-alkyl, C₂-C₇-alkenyl or C₂-C₇-alkynyl or R₄ represents C₂-C₇-alkanoyl; and X represents ligand (a),

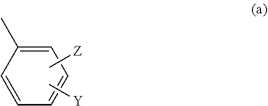

wherein Y may be in ortho, meta or para position and wherein Y represents carboxyl, C₁-C₇-alkoxy-carbonyl, aryloxycarbonyl, tetrazolyl, SO₃H or P(O)(OH)₂; and wherein Z represents hydrogen or a substituent selected from the group consisting of C₁-C₇-alkyl, C₁-C₇-alkoxy, halogen, CF₃, cyano and NO₂, as described in WO-A-2004/089916 or any of the specific RXR inhibitor of the above formula, as described in WO-A-2004/089916.

The present invention also provides a method of increasing tissue regeneration in a subject in need thereof, said method comprising the step of administering to said subject a therapeutically effective amount of an inhibitor of the retinoic acid pathway. The tissue can be epithelium, for instance intestinal epithelium. The tissue might have been damaged by irradiation. In another embodiment, the tissue is liver.

In some embodiments, the antagonist of the retinoic acid pathway is an antagonist of the RXR. Said antagonist can be selected from the group comprising HX 531, PA452 and UVI3003.

In other embodiments of the method of the invention, the antagonist of the retinoic acid pathway can a compound of formula (I) or a pharmaceutically acceptable salt thereof,

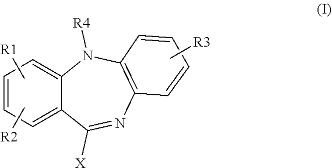

wherein R₁ and R₂, independently of each other, represent hydrogen, or C₁-C₇-alkyl, or R₁ and R₂ together with the carbon atoms of the phenyl ring to which they bind form a 5-, 6- or 7-membered cycloalkyl ring, which ring may optionally be substituted by one or more C₁-C₇-alkyl groups, which alkyl groups may also together form one or more 3-, 4-, 5-, 6- or 7-membered rings; R₃ represents —CN, —CO—R₅, or hydrogen, provided that, if R₃ is hydrogen, R₄ must represent C₃-C₇-alkenyl or C₃-C₇-alkynyl; R₅ represents aryl, or alkyl being unsubstituted or substituted by halogen, cyano, nitro, hydroxy, C₁-C₇-alkoxy, carboxyl or aryl; R₄ represents C₁-C₇-alkyl, C₂-C₇-alkenyl or C₂-C₇-alkynyl or R₄ represents C₂-C₇-alkanoyl; and X represents ligand (a),

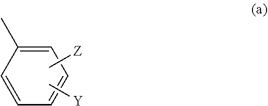

wherein Y may be in ortho, meta or para position and wherein Y represents carboxyl, C₁-C₇-alkoxy-carbonyl, aryloxycarbonyl, tetrazolyl, SO₃H or P(O)(OH)₂; and wherein Z represents hydrogen or a substituent selected from the group consisting of C₁-C₇-alkyl, C₁-C₇-alkoxy, halogen, CF₃, cyano and NO₂, as described in WO-A-2004/089916 or any of the specific RXR inhibitor of the above formula, as described in WO-A-2004/089916.

### BRIEF DESCRIPTION OF THE DRAWINGS

FIG. 1 RXR activity is indispensable for symmetry breaking and cell type balance maintenance Representative images of organoids cultured from single cells in indicated treatment conditions (left), composite maximum intensity projections of confocal z-stacks, nuclei, DAPI, antibody staining for Lysozyme and Aldolase B, scale bar, 50 μm. Violin plots showing distribution of mean Aldolase B staining intensity in control and treated organoids (top right). Box plots inside violin plots show quartile range, whiskers show value interval with excluded outliers, white dots indicate median values, n, number of individual organoids in respective conditions. DMSO, DMSO control, RXRi, 5 μM RXR antagonist. Bar plot indicating abundance of the enterocyst phenotype in indicated conditions (bottom right), values are percentages in individual wells, n of 3 for 10 μM atRA condition, n of 6 for DMSO control shown as mean±s.d.

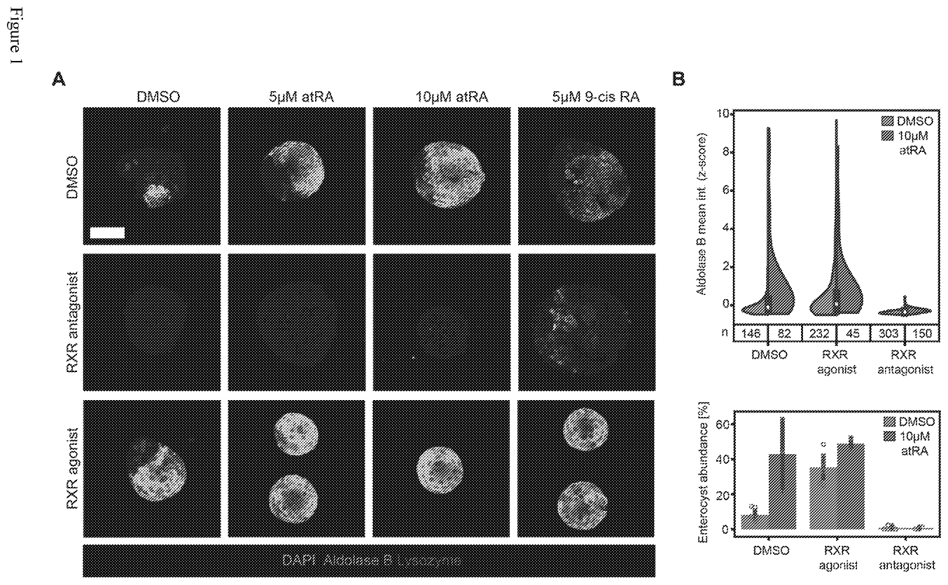

FIG. 2 RXR antagonist treatment induces a switch to regenerative fetal-like state and improves intestine regeneration in vivo. Body weight change over the course of the mouse study for the indicated treatment groups (n=6 in each group) calculated as area under curve (AUC) for the indicated treatment groups. Box plots show quartile range, whiskers show value interval with excluded outliers, white dots indicate median values. Two-sided t-test, p values as indicated.

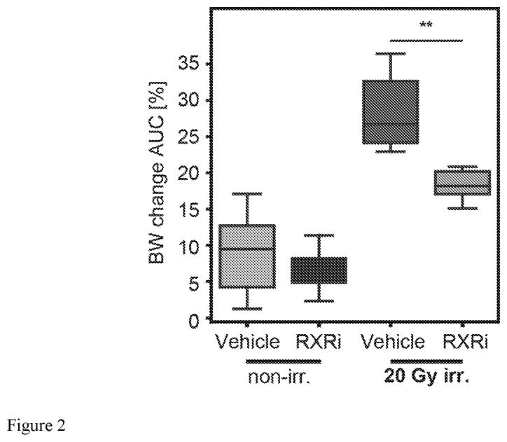

### DETAILED DESCRIPTION OF THE INVENTION

Using high-content image-based screening assay to unravel molecular mechanisms orchestrating organoid formation and self-organization, the present inventors serendipitously found a crucial role of retinoic acid nuclear receptors in controlling the exit from the regenerative state and in driving differentiation.

The present invention thus provides an inhibitor of the retinoic acid pathway for use in a method of increasing tissue regeneration. This tissue can be epithelium, for instance intestinal epithelium. In other embodiments, the tissue can be liver.

The inhibitor of the retinoic acid pathway can be an antagonist of the RXR. For instance, the antagonist of the retinoic acid pathway can selected from the group comprising HX 531, PA452 and UVI3003. In an embodiment, the inhibitor of the retinoic acid pathway is a compound of formula (I) or a pharmaceutically acceptable salt thereof,

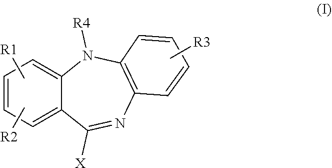

wherein R₁ and R₂, independently of each other, represent hydrogen, or C₁-C₇-alkyl, or R₁ and R₂ together with the carbon atoms of the phenyl ring to which they bind form a 5-, 6- or 7-membered cycloalkyl ring, which ring may optionally be substituted by one or more C₁-C₇-alkyl groups, which alkyl groups may also together form one or more 3-, 4-, 5-, 6- or 7-membered rings; R₃ represents —CN, —CO—R₅, or hydrogen, provided that, if R₃ is hydrogen, R₄ must represent C₃-C₇-alkenyl or C₃-C₇-alkynyl; R₅ represents aryl, or alkyl being unsubstituted or substituted by halogen, cyano, nitro, hydroxy, C₁-C₇-alkoxy, carboxyl or aryl; R₄ represents C₁-C₇-alkyl, C₂-C₇-alkenyl or C₂-C₇-alkynyl or R₄ represents C₂-C₇-alkanoyl; and X represents ligand (a),

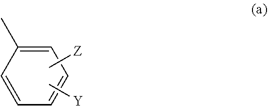

wherein Y may be in ortho, meta or para position and wherein Y represents carboxyl, C₁-C₇-alkoxy-carbonyl, aryloxycarbonyl, tetrazolyl, SO₃H or P(O)(OH)₂; and wherein Z represents hydrogen or a substituent selected from the group consisting of C₁-C₇-alkyl, C₁-C₇-alkoxy, halogen, CF₃, cyano and NO₂, as described in WO-A-2004/089916 or any of the specific RXR inhibitor of the above formula, as described in WO-A-2004/089916.

The present invention also provides a method of increasing tissue regeneration in a subject in need thereof, said method comprising the step of administering to said subject a therapeutically effective amount of an inhibitor of the retinoic acid pathway. The tissue can be epithelium, for instance intestinal epithelium. The tissue might have been damaged by irradiation. In another embodiment, the tissue is liver.

In some embodiments, the antagonist of the retinoic acid pathway is an antagonist of the RXR. Said antagonist can be selected from the group comprising HX 531, PA452 and UVI3003.

In other embodiments of the method of the invention, the antagonist of the retinoic acid pathway can a compound of formula (I) or a pharmaceutically acceptable salt thereof,

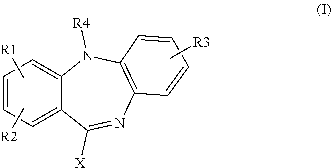

wherein R₁ and R₂, independently of each other, represent hydrogen, or C₁-C₇-alkyl, or R₁ and R₂ together with the carbon atoms of the phenyl ring to which they bind form a 5-, 6- or 7-membered cycloalkyl ring, which ring may optionally be substituted by one or more C₁-C₇-alkyl groups, which alkyl groups may also together form one or more 3-, 4-, 5-, 6- or 7-membered rings; R₃ represents —CN, —CO—R₅, or hydrogen, provided that, if R₃ is hydrogen, R₄ must represent C₃-C₇-alkenyl or C₃-C₇-alkynyl; R₅ represents aryl, or alkyl being unsubstituted or substituted by halogen, cyano, nitro, hydroxy, C₁-C₇-alkoxy, carboxyl or aryl; R₄ represents C₁-C₇-alkyl, C₂-C₇-alkenyl or C₂-C₇-alkynyl or R₄ represents C₂-C₇-alkanoyl; and X represents ligand (a),

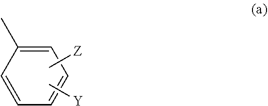

wherein Y may be in ortho, meta or para position and wherein Y represents carboxyl, C₁-C₇-alkoxy-carbonyl, aryloxycarbonyl, tetrazolyl, SO₃H or P(O)(OH)₂; and wherein Z represents hydrogen or a substituent selected from the group consisting of C₁-C₇-alkyl, C₁-C₇-alkoxy, halogen, CF₃, cyano and NO₂, as described in WO-A-2004/089916 or any of the specific RXR inhibitor of the above formula, as described in WO-A-2004/089916.

Selected terms are defined below and throughout the application. Compounds of the present invention are described using standard nomenclature. Unless defined otherwise, all technical and scientific terms used herein have the same meaning as is commonly understood by one of skill in the art to which this invention belongs. The following general definitions shall apply in this specification, unless otherwise specified:

As used herein, the terms “a” and “an” and “the” and similar references in the context of describing the invention are to be construed to cover both the singular and the plural, unless otherwise indicated herein or clearly contradicted by context. Where the plural form is used for compounds, salts, and the like, this is taken to mean also a single compound, salt, or the like.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values. When describing a dosage herein as “about” a specified amount, the actual dosage can vary by up to 10% from the stated amount: this usage of “about” recognizes that the precise amount in a given dosage form may differ slightly from an intended amount for various reasons without materially affecting the in vivo effect of the administered compound.

The terms “comprising” and “including” are used herein in their open-ended and non-limiting sense unless otherwise noted.

As used herein, the terms “subject” and “patient” are used herein interchangeably to refer to an animal (e.g., cow, horse, sheep, pig, chicken, turkey, quail, cat, dog, mouse, rat, rabbit, guinea pig, etc.), preferably a mammal such as a non-primate and a primate (e.g., monkey and human), most preferably a human. In certain embodiments, the patient is an embryo, fetus, infant, child, adolescent or adult.

An “inhibitor” or “antagonist” is a substance that decreases the rate of, or prevents, a reaction.

“Tissue regeneration” is the process of renewal and growth to repair or replace tissue that is damaged, e.g. by irradiation, or suffers from a disease.

As used herein, the term “subject in need of the treatment” refers to a subject needing regeneration of tissue. An appropriately qualified person is able to identify such an individual in need of treatment using standard behavioral testing protocols/guidelines. The same behavioral testing protocols/guidelines can also be used to determine whether there is improvement to the individual's disorder and/or symptoms.

The phrase “subject in need of such treatment” as used herein refers to a patient who displays symptoms or who will otherwise benefit from the described treatment, including, without limitation, one who (i) will receive treatment with the composition of the invention; (ii) is receiving the composition of the invention; or (iii) has received the composition of the invention. In some other embodiments, the phrase “subject in need of such treatment” also is used to refer to a patient who (i) will suffer from tissue damage; (ii) is suffering from tissue damage; or (iii) has suffered from tissue damage. In some other embodiments, the phrase “subject in need of such treatment” also is used to refer to a patient who (i) will be administered a composition of the invention; (ii) is receiving a composition of the invention; or (iii) has received a composition of the invention, unless the context and usage of the phrase indicates otherwise.

As used herein, the term “treatment” refers to a clinical intervention made in response to a disease, disorder or physiological condition manifested by a patient, particularly a patient suffering from tissue damages, e.g. due to irradiation. The aim of treatment may include, but is not limited to, one or more of the alleviation or prevention of symptoms, slowing or stopping the progression or worsening of a disease, disorder, or condition and the remission of the disease, disorder or condition. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. For example, in some embodiments treatment may improve behavioral performance of the subject. As used herein, the term “prevention” refers to any activity that reduces the burden of the individual later expressing those behavioral symptoms. This takes place at primary, secondary and tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications.

As used herein the term “agent” is understood to mean a substance that produces a desired effect in a tissue, system, animal, mammal, human, or other subject. It is also to be understood that an “agent” may be a single compound or a combination or composition of two or more compounds.

The pharmaceutical compositions of the invention may include a “therapeutically effective amount”, “effective amount” or a “prophylactically effective amount” of a compound described herein. The term “pharmaceutically effective amount”, “therapeutically effective amount” or “clinically effective amount” of a single therapeutic agent or of a combination of therapeutic agents is an amount sufficient, at dosages and for periods of time necessary, to provide an observable or clinically significant improvement over the baseline of clinically observable signs and symptoms of the disorders treated with the combination. A therapeutically effective amount may vary according to factors such as the disease state, age, sex, and weight of the individual. A therapeutically effective amount is also one in which any toxic or detrimental effects of the therapeutic agents are outweighed by therapeutically beneficial effects. A “therapeutically effective dosage” preferably modulates a measurable parameter in a desired manner. The ability of a compound to desirably modulate a measurable parameter can be evaluated in an animal model system predictive of efficacy in humans to help establish suitable dosing levels and schedules. Alternatively, this property of a composition can be evaluated by examining the ability of the compound to modulate an undesired parameter by using in vitro assays known to the skilled practitioner.

As used herein, a “therapeutic protocol” or “prophylactic protocol” refers to a regimen of timing and dosing of one or more therapies. A used herein, a “protocol” includes dosing schedules and dosing regimens. It may include executing a protocol, which may include administering one or more drugs to a patient (human or otherwise), in an effort to alleviate signs or symptoms of the disease. In certain embodiments, therapeutic treatment prevents worsening of a disease or condition.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Within the meaning of the present disclosure, the term “protect” is used herein to mean prevent, delay, or treat, or all, as appropriate, development, continuance or aggravation of a disease in a subject, e.g., a mammal or human. The term “prevent”, “preventing” or “prevention” as used herein comprises the prevention of at least one symptom associated with or caused by the state, disease or disorder being prevented.

The term “inhibition”, “inhibitor,” or “antagonist” includes a reduction in a certain parameter, e.g., an activity, of a given molecule or pathway.

Retinoids comprise a group of compounds each composed of three basic parts: a trimethylated cyclohexene ring that is a bulky hydrophobic group, a conjugated tetraene side chain that functions as a linker unit, and a polar carbon-oxygen functional group. Biochemical conversion of carotenoid or other retinoids to retinoic acid (RA) is essential for normal regulation of a wide range of biological processes including development, differentiation, proliferation, and apoptosis. Retinoids regulate various physiological outputs by binding to nuclear receptors called retinoic acid receptors (RARs) and retinoid X receptors (RXRs), which themselves are DNA-binding transcriptional regulators. The functional response of RA and their receptors are modulated by a host of coactivators and corepressors. Retinoids are essential in the development and function of several organ systems; however, deregulated retinoid signaling can contribute to serious diseases.

RXR and RAR are nuclear receptors activated by ligand binding and are able to translate changes in ligand abundance directly to gene expression (Evans and Mangelsdorf, 2014). RXR-RAR heterodimer in particular has been shown to bind regions of DNA containing retinoic acid response elements (RAREs) and to mediate repression of target gene expression in ligand-unbound apo-state (Rochel and Moras, 2014). The heterodimer is activated by binding of a vitamin A metabolite all-trans retinoic acid (atRA) to RAR mediating a conformation change to holo-state that drives recruitment of co-activator machinery and ultimately activates target gene expression.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

### Examples

The Examples below are set forth to aid in the understanding of the invention but are not intended, and should not be construed, to limit its scope in any way.

In the context of intestinal epithelium homeostasis a number of downstream effects of retinoic acid signaling have been proposed, including transcriptional induction of HOXA5 and downstream negative regulators of the cell cycle (Ordonez-Moran et al., 2015). Induction of these genes has been shown to be triggered by activated RXR-RAR heterodimer The present inventors observed that RXR antagonists have a highly penetrant phenotype marked by absence of differentiation. They also observed that, conversely, organoids cultured in presence of an RXR agonist and all-trans retinoic acid (atRA) displayed an increase in absorptive enterocytes. Vitamin A (retinol), the precursor to atRA cannot be synthesized de-novo by mammals and is taken up from the digestive system (Borel et al., 2001). However, retinol can also be metabolized by intestinal cells to form the signaling active atRA (Lampen et al., 2000). Analysis of a single cell RNA sequencing dataset (Haber et al., 2017) identified genes involved in retinol metabolism as marker genes of enterocytes. Among them is ALDH1A1 (formerly RALDH1), the ALDH1 isoform expressed in adult tissue that has previously been reported to be enriched in differentiated cells of the small intestine (Goverse et al., 2017). As ALDH1A1-mediated conversion of retinyl aldehyde is the rate-limiting step in atRA production (Molotkov and Duester, 2003), enterocytes in the villi are potentially able to maintain higher intracellular atRA levels compared to crypt cells. The inventors validated their hypothesis with ALDH1A1 antibody staining, observing exclusive localization of ALDH1A1 to the cells outside the crypt region and observing upregulation of RARE reporter gene in the cells outside the crypt Treatment with atRA resulted in expansion of the ALDH1A1-positive as well as RARE-positive region.

As atRA is a metabolite of vitamin A, The inventors tested whether removing the metabolic supply of atRA precursor would affect enterocyte differentiation and cultured organoids in presence of ALDH1A1 inhibitor observing decreased enterocyte differentiation stemming from an increase in cycling cells.

The inventors observed that at the gene expression level, RXR antagonist-treated organoids retain a fetal-like regenerative transcriptional signature, making RXR antagonist a potential tool to promote regeneration.

To validate the regeneration-promoting effect of RXR antagonist treatment, the inventors devised an in vivo study using an irradiation-induced colitis model. Small intestine of mice was irradiated with 20Gy to ablate cycling cells, subsequently mice were left to recover while treated with RXR antagonist. Mice were monitored for 6 days after irradiation and body weight was used to quantitatively describe the changes in recovery process. RXR antagonist treatment significantly improved regeneration of the intestine post-irradiation resulting in less weight loss in the treated mice compared to irradiated controls. As irradiation-induced damage increases the permeability of the intestinal epithelial barrier, the inventors assessed the amount of blood in the stool of the mice belonging to each treatment cohort observing lower hemoccult in RXR treated mice compared to irradiated controls and lower compound concentration in serum after 5 days of recovery. These observations indicate an improvement in the barrier function of the intestine. At the tissue level, in irradiated mice, RXR antagonist treatment resulted in less decellularization, increased villus length and functional villi containing mature goblet cells.

The inventors thus found that inhibition of RXR-mediated signaling can be used to improve regeneration.

### REFERENCES

Chacon-Martinez, C. A., Koester, J., and Wickstrom, S. A. (2018). Signaling in the stem cell niche: regulating cell fate, function and plasticity. Development 145.
Farin, H. F., Van Es, J. H., and Clevers, H. (2012). Redundant sources of Wnt regulate intestinal stem cells and promote formation of Paneth cells. Gastroenterology 143, 1518-1529 e1517
Evans, R. M., and Mangelsdorf, D. J. (2014). Nuclear Receptors, RXR, and the Big Bang. Cell 157, 255-266.
Farin, H. F., Van Es, J. H., and Clevers, H. (2012). Redundant sources of Wnt regulate intestinal stem cells and promote formation of Paneth cells. Gastroenterology 143, 1518-1529 e1517
Grun, D., Lyubimova, A., Kester, L., Wiebrands, K., Basak, O., Sasaki, N., Clevers, H., and van Oudenaarden, A. (2015). Single-cell messenger RNA sequencing reveals rare intestinal cell types. Nature 525, 251-255.
Qi, Z., Li, Y., Zhao, B., Xu, C., Liu, Y., Li, H., Zhang, B., Wang, X., Yang, X., Xie, W., et al. (2017). BMP restricts stemness of intestinal Lgr5(+) stem cells by directly suppressing their signature genes. Nat Commun 8, 13824
Sato, T., van Es, J. H., Snippert, H. J., Stange, D. E., Vries, R. G., van den Born, M., Barker, N., Shroyer, N. F., van de Wetering, M., and Clevers, H. (2011). Paneth cells constitute the niche for Lgr5 stem cells in intestinal crypts. Nature 469, 415-418.
Sato, T., Vries, R. G., Snippert, H. J., van de Wetering, M., Barker, N., Stange, D. E., van Es, J. H., Abo, A., Kujala, P., Peters, P. J., et al. (2009). Single Lgr5 stem cells build crypt-villus structures in vitro without a mesenchymal niche. Nature 459, 262-265.
Sato, Y., Ramalanjaona, N., Huet, T., Potier, N., Osz, J., Antony, P., Peluso-Iltis, C., Poussin-Courmontagne, P., Ennifar, E., Mely, Y., et al. (2010). The “Phantom Effect” of the Rexinoid LG100754: structural and functional insights. PLoS One 5, el5119.
Serra, D., Mayr, U., Boni, A., Lukonin, I., Rempfler, M., Challet Meylan, L., Stadler, M. B., Strnad, P., Papasaikas, P., Vischi, D., et al. (2019). Self-organization and symmetry breaking in intestinal organoid development. Nature 569, 66-72.

1. An inhibitor of the retinoic acid pathway for use in a method of increasing tissue regeneration.

2. The inhibitor of claim 1 wherein the tissue is epithelium.

3. The inhibitor of claim 2 wherein the tissue is intestinal epithelium.

4. The inhibitor of claim 1 wherein the tissue is liver.

5. The inhibitor of claim 1 wherein the antagonist of the retinoic acid pathway is an antagonist of the RXR.

6. The inhibitor of claim 1 wherein the antagonist of the retinoic acid pathway is selected from the group comprising HX 531, PA452 and UVI3003.

7. The inhibitor of claim 1 wherein the antagonist of the retinoic acid pathway is a compound of formula (I) or a pharmaceutically acceptable salt thereof, 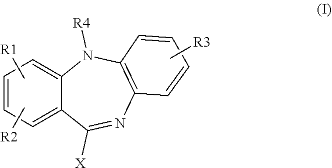
wherein R₁ and R₂, independently of each other, represent hydrogen, or C₁-C₇-alkyl, or R₁ and R₂ together with the carbon atoms of the phenyl ring to which they bind form a 5-, 6- or 7-membered cycloalkyl ring, which ring may optionally be substituted by one or more C₁-C₇-alkyl groups, which alkyl groups may also together form one or more 3-, 4-, 5-, 6- or 7-membered rings; R₃ represents —CN, —CO—R₅, or hydrogen, provided that, if R₃ is hydrogen, R₄ must represent C₃-C₇-alkenyl or C₃-C₇-alkynyl; R₅ represents aryl, or alkyl being unsubstituted or substituted by halogen, cyano, nitro, hydroxy, C₁-C₇-alkoxy, carboxyl or aryl; R₄ represents C₁-C₇-alkyl, C₂-C₇-alkenyl or C₂-C₇-alkynyl or R₄ represents C₂-C₇-alkanoyl; and X represents ligand (a), 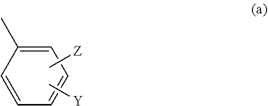
wherein Y may be in ortho, meta or para position and wherein Y represents carboxyl, Cₜ-C₇-alkoxy-carbonyl, aryloxycarbonyl, tetrazolyl, SO₃H or P(O)(OH)₂; and wherein Z represents hydrogen or a substituent selected from the group consisting of C₁-C₇-alkyl, C₁-C₇-alkoxy, halogen, CF₃, cyano and NO₂.

8. A method of increasing tissue regeneration in a subject in need thereof, said method comprising the step of administering to said subject a therapeutically effective amount of an inhibitor of the retinoic acid pathway.

9. The method of claim 8 wherein the tissue is epithelium.

10. The method of claim 9 wherein the tissue is intestinal epithelium.

11. The method of claim 8 wherein said tissue has been damaged by irradiation.

12. The method of claim 8 wherein the tissue is liver.

13. The inhibitor of claim 8 wherein the antagonist of the retinoic acid pathway is an antagonist of the RXR.

14. The inhibitor of claim 8 wherein the antagonist of the retinoic acid pathway is selected from the group comprising HX 531, PA452 and UVI3003.

15. The inhibitor of claim 8 wherein the antagonist of the retinoic acid pathway is a compound of formula (I) or a pharmaceutically acceptable salt thereof, 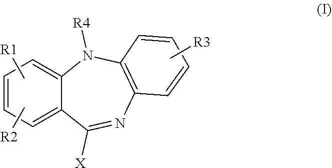
wherein R₁ and R₂, independently of each other, represent hydrogen, or C₁-C₇-alkyl, or R₁ and R₂ together with the carbon atoms of the phenyl ring to which they bind form a 5-, 6- or 7-membered cycloalkyl ring, which ring may optionally be substituted by one or more C₁-C₇-alkyl groups, which alkyl groups may also together form one or more 3-, 4-, 5-, 6- or 7-membered rings; R₃ represents —CN, —CO—R₅, or hydrogen, provided that, if R₃ is hydrogen, R₄ must represent C₃-C₇-alkenyl or C₃-C₇-alkynyl; R₅ represents aryl, or alkyl being unsubstituted or substituted by halogen, cyano, nitro, hydroxy, C₁-C₇-alkoxy, carboxyl or aryl; R₄ represents C₁-C₇-alkyl, C₂-C₇-alkenyl or C₂-C₇-alkynyl or R₄ represents C₂-C₇-alkanoyl; and X represents ligand (a), 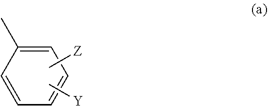
wherein Y may be in ortho, meta or para position and wherein Y represents carboxyl, C₁-C₇-alkoxy-carbonyl, aryloxycarbonyl, tetrazolyl, SO₃H or P(O)(OH)₂; and wherein Z represents hydrogen or a substituent selected from the group consisting of C₁-C₇-alkyl, C₁-C₇-alkoxy, halogen, CF₃, cyano and NO₂.
